(19) United States
(12) Patent Application Publication
(10) Pub. No.: US 2014/0120149 A1
(43) Pub. Date: May 1, 2014

(54) CALCIUM SULPHATE BASED COMPOSITE

(71) Applicant: THE ROYAL INSTITUTION FOR THE ADVANCEMENT OF LEARNING/MCGILL UNIVERSITY, Montreal (CA)

(72) Inventors: ANDREW FLYNN, TORONTO (CA); J. DENNIS BOBYN, MOSMAN (AU); MICHAEL TANZER, HAMPSTEAD (CA); JAKE E. BARRALET, MONTREAL (CA)

(73) Assignee: THE ROYAL INSTITUTION FOR THE ADVANCEMENT OF LEARNING/MCGILL UNIVERSITY, Montreal (CA)

(21) Appl. No.: 14/067,237

(22) Filed: Oct. 30, 2013

Related U.S. Application Data
(60) Provisional application No. 61/719,997, filed on Oct. 30, 2012.

(51) Int. Cl. A61L 27/12; A61L 27/54; A61L 27/42

(52) U.S. Cl. CPC A61L 27/12; A61L 27/427; A61L 27/54
USPC 424/423; 424/602

(57) ABSTRACT

A composite comprising monetite and calcium sulphate is provided.

### CROSS REFERENCE TO RELATED APPLICATIONS

This application claims benefit, under 35 U.S.C. §119(e), of U.S. provisional application Ser. No. 61/719,997, filed on Oct. 30, 2012.

### FIELD OF THE INVENTION

The present invention relates to calcium sulphate based composites. More specifically, the present invention is concerned with such composites for use as bone graft substitutes.

### BACKGROUND OF THE INVENTION

Trauma to the skeleton can often necessitate the use of grafts to fill volumes of missing bone or to enhance healing of severe fractures. For example, revision arthroplasty surgery often involves loss of bone stock and cavity defects that result in suboptimal implant fit, reduced initial stability, and reduced potential for biologic fixation of porous implants. Currently the materials most commonly used to in bone grafting are morcelized autograft, allograft and industrially processed demineralized human bone. Such porous grafts are commonly used with segmental defects of long and flat bone fractures. Several types of biomaterials have been proposed for defect filling as substitutes for autogenous bone graft; these include calcium phosphate and calcium sulphate based materials. While there have been attempts to use such synthetic materials, they have normally been combined with autograft to act as ‘expanders’ to make less autograft go further. In a recent review of human studies the authors conclude: “Whilst current bone graft substitute materials provide some degree of osseoconduction, osseoinduction and substitution, this is generally less than that observed with autograft or allograft.” [Beswick and Blom, Injury, 2011, S40-S46].

In addition to replacing missing bone, it is sometimes necessary to induce bone to form within pores of a material providing mechanical interlocking and fixation. Known as osseointegration Clearly the requirements of grafts for the purposes of replacing deficient bone stock and induding osseointegration are different, yet the same graft materials are often used for both applications.

### BRIEF DESCRIPTION OF THE DRAWINGS

In the appended drawings:

FIG. 1 shows XRD patterns of granule material pre- and post-autoclaving. Brushite (*), Monetite (°);

FIG. 2 shows SEM images of the monetite granules, 212-500 μm A) and 500-1000 μm B);

FIG. 3 shows SEM of the monetite composite (60 wt % 212-500 μm granules P:L=2.0) surface showing granules within the calcium sulphate matrix;

FIG. 4 shows the effect of granule size on setting time A) and compressive strength B) for composites with 50 wt % granules and P:L=2.0 g/ml;

FIG. 5 shows the effect of weight percentage granules on setting time A) and compressive strength B) for composites made with 212-500 μm granules at a P:L=2.0 g/ml;

FIG. 6 shows the effect of composite P:L on setting time A) and compressive strength B) for composites made with 50 wt % 212-500 μm granules;

FIG. 7 shows the effect of weight percentage cement seeds on setting time A) and compressive strength B) for composites made with 50 wt % 212-500 μm granules at a P:L of 3.75 g/ml;

FIG. 8 shows A) implant with one proximal gap packed with CaS material, B) Pre-harvest radiograph;

FIG. 9 shows gap filling by both bone and graft materials at time zero (pale grey) and at 12 weeks (darker gray); compared with an empty gap.

FIG. 10 shows images from X-ray scans of CaP-filled gap. A) Longitudinal image at time zero. B) Longitudinal and C) transverse images at 12 weeks;

FIG. 11 shows images from X-ray scans of CaS-filled gap. A) Longitudinal image at time zero. B) Longitudinal and C) transverse images at 12 weeks;

FIG. 12 shows the extent of bone ingrowth (dark grey) and remaining graft and new bone around implants (pale grey) packed with either CaPO₄ (CaP, left) and a composite according to an embodiment of the invention (CaS) (right); and

FIG. 13 shows transverse BSEM images of A) CaP-filled implant and B) CaS-filled implant after 12 weeks (the darker grey is bone, lighter grey is residual CaS). Grey arrows show bond in the implant pores.

### DESCRIPTION OF THE INVENTION

In accordance with the present invention, there is provided:
1. A composite comprising monetite and calcium sulphate.
2. The composite of item 1, wherein the monetite is in the form of granules in a matrix of calcium sulphate.
3. The composite of item 2, where the granules range from about 10 to about 1500 μm in size.
4. The composite of item 3, where the granules range from about 200 to about 500 μm in size.
5. The composite of item 3 where the granules range from about 20 to about 400 μm in size.
6. The composite of any one of items 2 to 5, comprising between about 10 and about 80 wt % of granules, based on the total dry weight of the composite.
7. The composite of item 6, comprising between about 20 and about 60 wt % of granules, based on the total dry weight of the composite.
8. The composite of item 7, comprising between about 30 and about 50 wt % of granules, based on the total dry weight of the composite.
9. The composite of item 8, comprising about 50 wt % of granules, based on the total dry weight of the composite.
10. The composite of any one of items 1 to 9, further comprising an accelerant.
11. The composite of item 10, wherein the accelerant comprises seeds of calcium sulphate.
12. The composite of item 11, wherein the seeds are seeds of calcium sulphate dihydrate or dehydrate.
13. The composite of item 11 or 12, comprising between about 0.2 and about 3.0 wt % of seeds.
14. The composite of item 13, comprising about 1.0 wt % of seeds.
15. The composite any one of items 1 to 14, wherein the calcium sulphate is calcium sulphate hemihydrate in powder form, and forms a powder matrix around the monetite.
   It should be understood that when such composite is mixed with a curing liquid and allowed to set, the calcium sulphate hemihydrate will form a solid matrix of calcium sulphate dihydrate or dehydrate (if further dehydrated) around the monetite. In other words, it would form the composite of item 16 below.
16. The composite any one of items 1 to 14, wherein the calcium sulphate is calcium sulphate dihydrate or dehydrate in the form of a porous solid and forms a solid matrix around the monetite.
17. The composite any one of items 1 to 14, wherein the calcium sulphate is in powder form and wherein the calcium sulphate and the monetite are mixed with a curing liquid to form of a paste.
18. The composite of item 17, wherein paste comprises between about 1.5 and about 4.0 g of calcium sulfate/monetite per ml of curing liquid.
19. The composite of item 18, where the paste comprises between about 3.0 and about 4.0 g of calcium sulfate/monetite per ml of curing liquid
20. The composite of item 19, wherein the paste comprises 3.75 g of calcium sulfate/monetite per ml of curing liquid.
21. The composite of any one of items 17 or 20, wherein the curing liquid is saline or another non-toxic aqueous liquid.
   This liquid could be blood, for example.
22. A bone graft substitute made of the composite of any one of items 1 to 21.
23. A method for stimulating bone growth in a bone defect, the method comprising:
   i. providing a composite according to any one of items 15 to 21,
   ii. when the composite is a composite according to item 15, mixing the composite with a sterile curing liquid to form a paste,
   iii. applying the composite in or near the defect, and
   iv. when the composite is a composite according to item 15, or 17 to 21, allowing the composite to set.
24. A method for stimulating bone growth in a gap between a bone and an implant, in a gap between two bones, or in a gap in a single bone, the method comprising:
   i. providing a composite according to any one of items 15 to 21,
   ii. when the composite is a composite according to item 15, mixing the composite with a sterile curing liquid to form a paste,
   iii. applying the composite in or near the gap,
   iv. when the composite is a composite according to item 15, or 17 to 21, allowing the composite to set.
25. A method for enhancing healing of a bone-implant gap, the method comprising:
   i. providing a composite according to any one of items 15 to 21,
   ii. when the composite is a composite according to item 15, mixing the composite with a sterile curing liquid to form a paste,
   iii. applying the composite in the gap,
   iv. when the composite is a composite according to item 15, or 17 to 21, allowing the composite to set.
26. A method for enhancing osteointegration in a bone of a porous titanium bone implant, the method comprising:
   i. providing a composite according to any one of items 15 to 21,
   ii. when the composite is a composite according to item 15, mixing the composite with sterile curing liquid to form a paste,
   iii. applying the composite in a gap between the bone and the implant,
   iv. when the composite is a composite according to item 15, or 17 to 21, allowing the composite to set.
27. A method for promoting bone growth around an implant, the method comprising:
   i. providing a composite according to any one of items 15 to 21,
   ii. when the composite is a composite according to item 15, mixing the composite with sterile curing liquid to form a paste,
   iii. applying the composite around the implant,
   iv. when the composite is a composite according to item 15, or 17 to 21, allowing the mixture to set.
28. The method of claim 27, wherein the implant is a porous implant.
29. The method of any one of items 23 to 28, wherein the curing liquid is sterile saline.
30. The method any one of items 23 to 29, wherein paste comprises between about 1.5 and about 4.0 g of calcium sulfate/monetite per ml of curing liquid.
31. The method of item 30, where the paste comprises between about 3.0 and about 4.0 g of calcium sulfate/monetite per ml of curing liquid
32. The method of item 31, wherein the paste comprises 3.75 g of calcium sulfate/monetite per ml of curing liquid.

The inventors have previously developed an assay to quantify bone ingrowth into porous model implants and have now gathered data (shown below) indicating that the new synthetic bone graft of the invention exceeded the performance of an industry leading allograft material in a large animal model.

Monetite is a known mineral (CaHPO₄) consisting of an acid calcium hydrogen phosphate. It occurs in small quantities in many phosphate deposits, particularly as an incrustation on ancient bones and as a decomposition product of guano (seafowl excrement). It can be obtained from the thermal decomposition of brushite.

Calcium sulphate hemihydrate (CaSO₄.½H₂O), commonly known as “plaster of Paris”, can be produced by heating gypsum (CaSO₄.2H₂O). Crystacal Alpha K is a fine plaster (calcium sulphate hemihydrate) made by British Gypsum.

It is understood that in the above described methods, the wound of the patient should be closed and the bone should be given sufficient time to grow in the composite.

The use of the terms “a” and “an” and “the” and similar referents in the context of describing the invention (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise claimed.

No language in the specification should be construed as indicating any non-claimed element as essential to the practice of the invention.

Herein, the term “about” has its ordinary meaning. In embodiments, it may mean plus or minus 10% of the numerical value qualified.

Unless otherwise defined, all technical and scientific terms used herein have the same meaning as commonly understood by one of ordinary skill in the art to which this invention belongs.

Other objects, advantages and features of the present invention will become more apparent upon reading of the following non-restrictive description of specific embodiments thereof, given by way of example only with reference to the accompanying drawings.

### DESCRIPTION OF ILLUSTRATIVE EMBODIMENTS

The present invention is illustrated in further details by the following non-limiting examples.

Prior to the implantation study, considerable work was performed to optimize handling and setting behaviour of the composite of the invention and a set of properties were derived that were optimal compared with others trialed and were sufficient for surgery. The composite satisfied the surgical requirements of a moderately strong osteoconductive material that is easily handled and sets within a clinically acceptable time frame to allow the surgeon to apply the material without compromising strength or prolonging surgery. As shown below, electron microscopy of the composite fracture surface clearly showed granules within the cement matrix, forming an extremely porous solid, ideal for bone ingrowth. When the new composite was compared with a leading commercial synthetic bone graft substitute it was apparent that there was much more remaining graft AROUND the model porous implant coated with the positive control. Closer evaluation revealed that the extent of bone growth INTO the porous metal rod was much higher. This last capacity for bone growth into the rod is believed to be important to rapid implant stability. When these data were quantified, it was apparent that bone in-growth was nearly double for the new composite. Similar results were obtained with demineralized human bone allografts. These are quite extraordinary results indicative of a truly unique product of substantial benefit to significant numbers of future patients.

### Example 1

### Monetite-Calcium Sulphate Composite for Use in Revision Surgery

### Materials and Methods

### Materials

Crystacal Alpha K plaster (calcium sulphate hemihydrate—CaSO₄.½H₂O) was obtained from BPB (Valley Forge, Pa., USA) and monocalcium phosphate monohydrate (MCPM) was obtained from ACBR GmbH (Karlsruhe, Germany). Beta tricalcium phosphate (β-TCP) was synthesized from a stoichiometric mixture of calcium carbonate and dicalcium phosphate dihydrate obtained from Merck KGaA (Darmstadt, Germany) heated to 1400° C. for 14 h followed by 1000° C. for 6 h. The resulting material was ground until <160 μm in size.

### Methods

Brushite powder was made from β-TCP and MCPM mixed in a weight ratio of 1.5:1 β-TCP:MCPM mixed in a mortar and pestle. The powder mixture was mixed with de-ionized water at a powder-to-liquid ratio (P:L) of 3.0 g/ml and left to set overnight. After curing, the brushite was autoclaved at 121° C. for 20 minutes to induce the phase transformation to monetite. The monetite was characterized using X-ray diffractometry (XRD; Philips model PW1710, Bedrijven b. v. S&I, The Netherlands)

After autoclaving, the monetite was crushed into granules using a mortar and pestle and stratified using ASTM standard sieves (ASTM E-11 Specification No. 18, 35, 70) to create granules ranging in size from 212 to 500 μm and from 500 to 1000 μm.

Monetite granules were mixed in with Crystacal Alpha K to determine the optimal mixture for handling. The effects of granule size, weight percentage granules, powder-to-liquid ratio (P:L) and weight percentage calcium sulphate cement seeds on the setting time and compressive strength were measured to determine the optimal mixture. Qualitative results were also obtained for the ease of handling of the mixtures.

The composites were mixed and placed in cylindrical PTFE mould forms (6 mm ø×12 mm) and the setting time of the mixtures was determined using Gilmore needles (ASTM C266-08). The mechanical strength of the set composites was tested 24 h after mixing using a universal testing machine (Instron 5569, Norwood, Mass., USA).

Surface morphology of the cement granules and set composite were examined using SEM (Hitachi S-4700 Field Emission STEM, Schaumburg, Ill., USA)

### Results

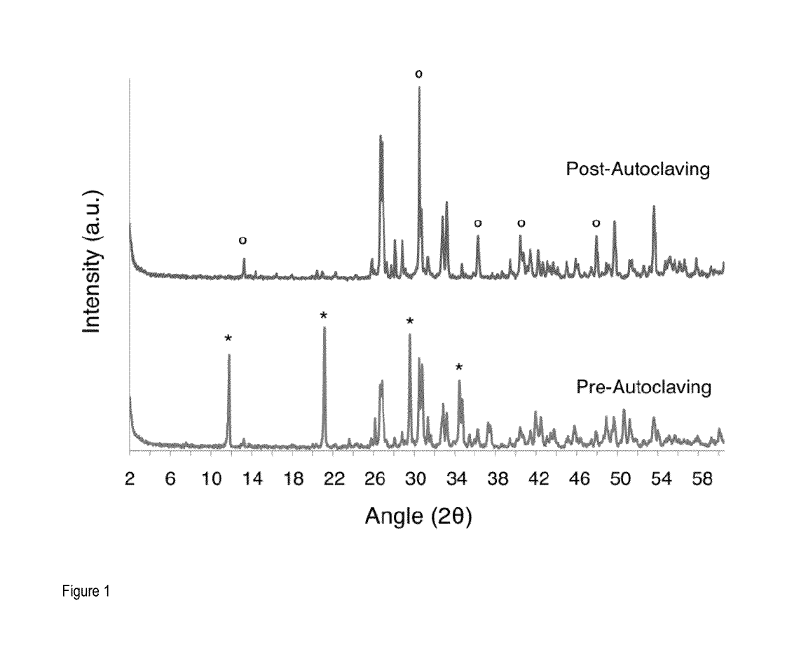

XRD analysis, shown in FIG. 1, confirmed the granule materials pre- and post-autoclaving were brushite and monetite, respectively.

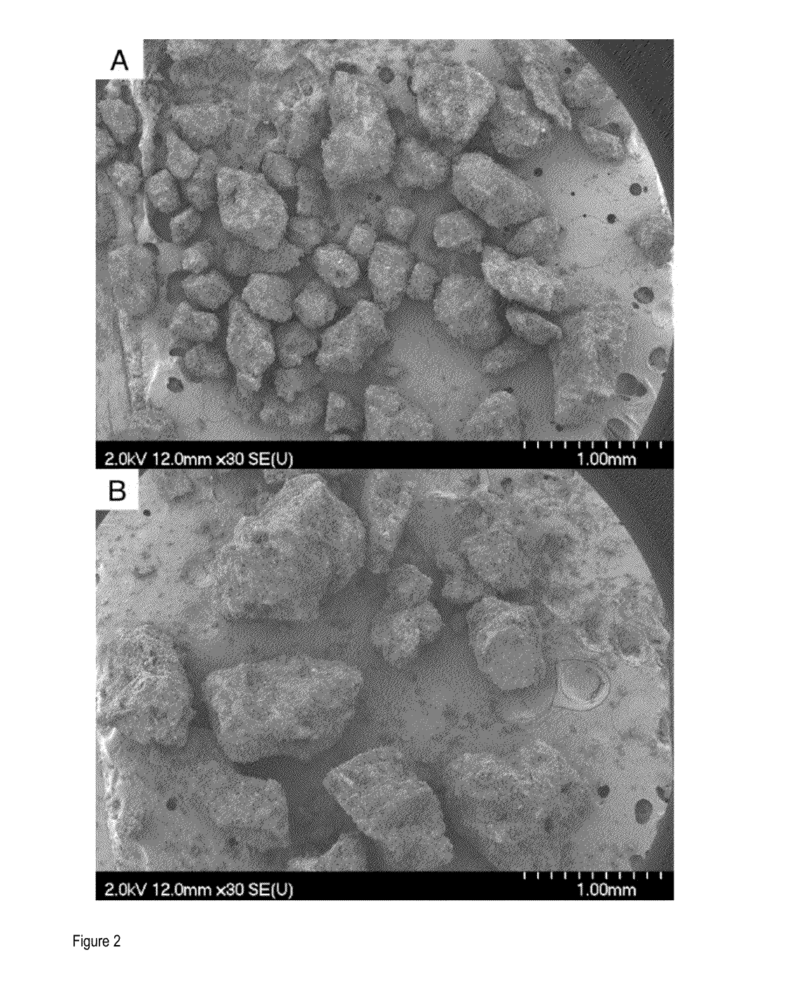

SEM imaging of the monetite granules showed a distribution of sizes within each range (FIG. 2). Larger granules in the 212-500 μm range showed oblong shapes with some appearing to be larger than 500 μm in one direction having fallen through the sieve in a favourable orientation (FIG. 2A). The inclusion of these larger granules should be in a low percentage and have no effect on the composites as a whole.

The various parameters of the composites were adjusted to measure their effects on both the qualitative and quantitative properties of the material. The properties tested are shown in Table 1.

TABLE 1
Tests and parameters for determining the properties of the composites

| Test | Values | Constant Parameters |
|---|---|---|
| Effect of Granule Size | 212-500 μm, 500-100 μm | Weight Percentage Granules: 50 wt % |
| | | P:L = 2.0 g/ml |
| Effect of Weight Percentage | 20, 30, 40, 50, 60 wt % Granules | Granule Size: 212-500 μm |
| Granules (Total mass) | | P:L = 2.0 g/ml |
| Effect of Powder-to-Liquid (P:L) | 2.0, 3.0, 3.5, 4.0 g/ml | Granule Size: 212-500 μm |
| Ratio | | Weight Percentage Granules: 50 wt % |
| Effect of Cement Seeds | 0.5, 1.0. 1.5 wt % | Granule Size: 212-500 μm |
| | | Weight Percentage Granules: 50 wt % |
| | | P:L = 4.0 g/ml |

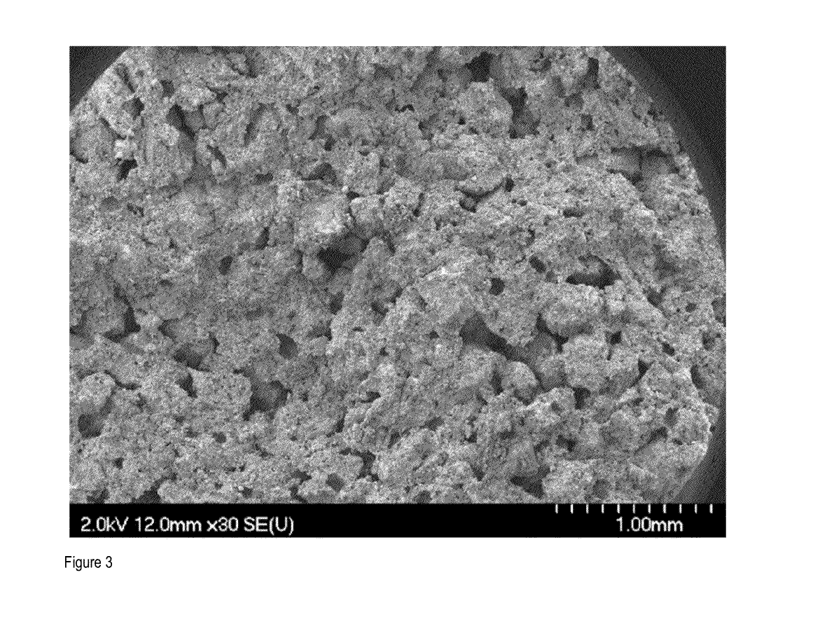

SEM imaging of the composite fracture surface clearly showed monetite granules within the calcium sulphate matrix, forming an extremely porous solid (FIG. 3). Note the inherent microporosity.

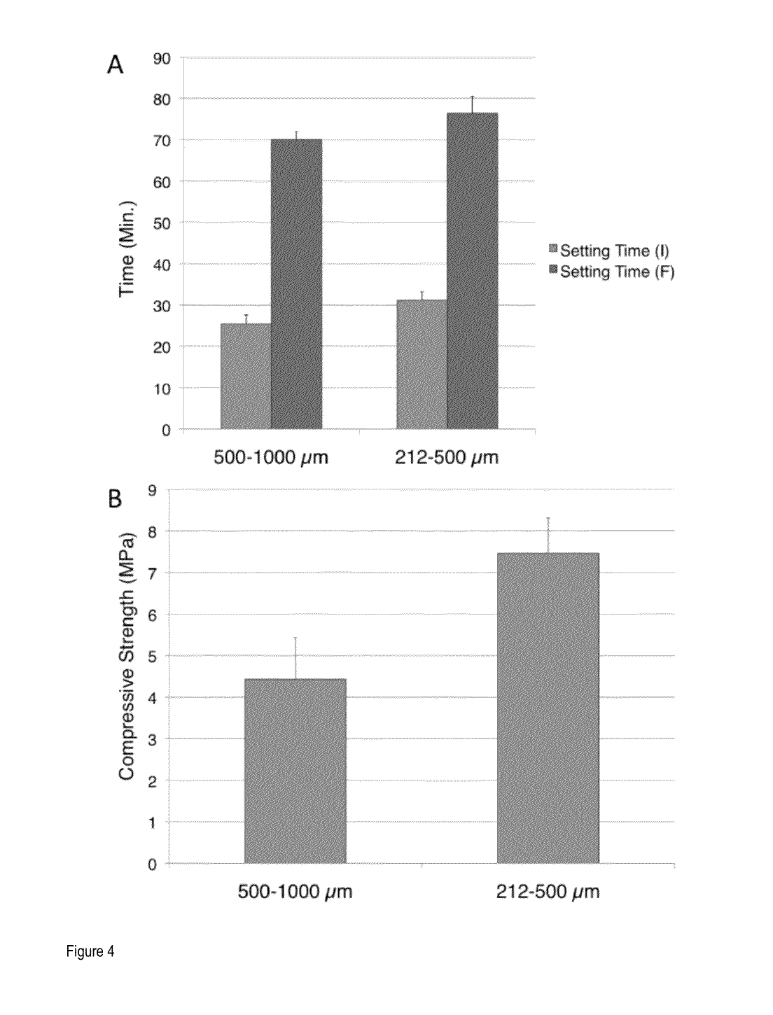

The effect of granule size on the properties of the composites was mixed. Upon mixing, it was qualitatively observed that the 212-500 μm granules allowed for a more mixable paste that was easier to form into the mould. The ease of handling of the composite had little effect on the setting time with both mixtures setting. The 212-500 μm mixture was however slightly slower (FIG. 4A). The effect on compressive strength was more defined, with the 212-500 μm granules forming a composite approximately 3 MPa stronger than that of the 500-1000 μm granules.

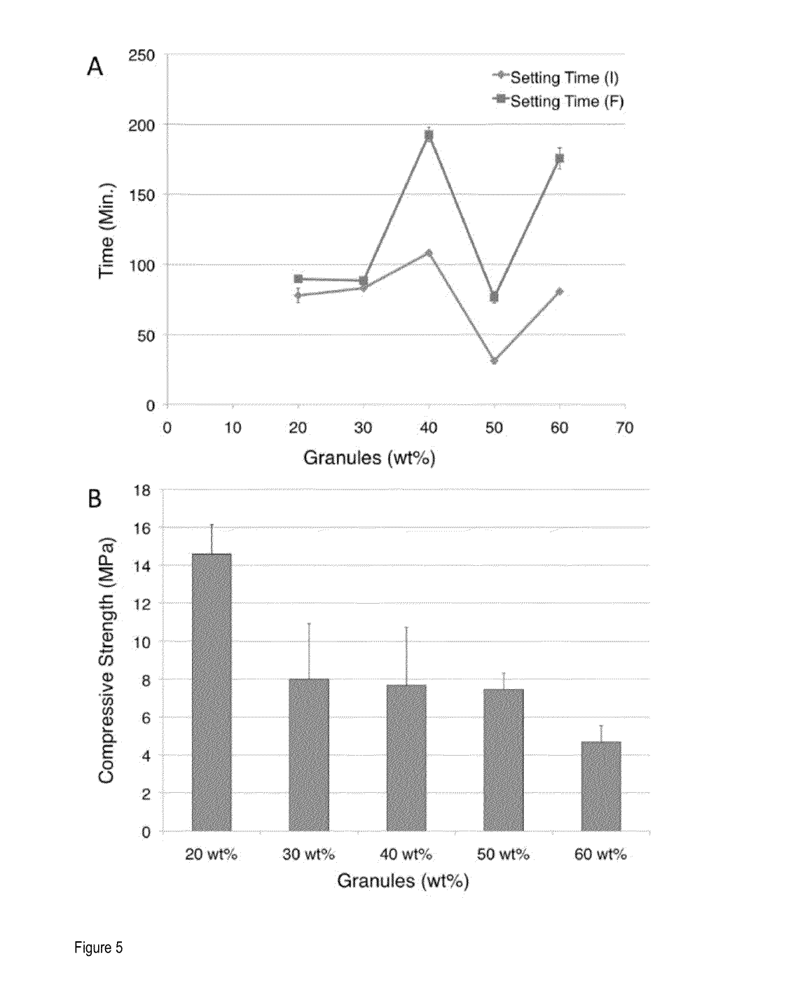

The effect of the weight percentage granules showed a mixed relationship for the initial (I) and final (F) setting times, with a large amount of fluctuation and no clear trend (FIG. 5A). The strength of the materials however was found to have a clear declining trend with the increase in granules. The decline showed a sharp drop from 20 wt % where it remained steady between 30-50 wt % before dropping again at 60 wt % (FIG. 5B). Increases in the weight percentage of granules resulted in progressively more difficult mixing. Due to the low flow of the granules, the mixtures were harder to apply pressure to and ensure uniform liquid phase distribution. At 60 wt % granules, the granules made it difficult to form the mixture into the mould and also resulted in a grainy cement appearance post-loading. The grainy appearance manifested itself as a porous structure. The cured material had poor handling properties with whole granules flaking off near the bottom of the composites.

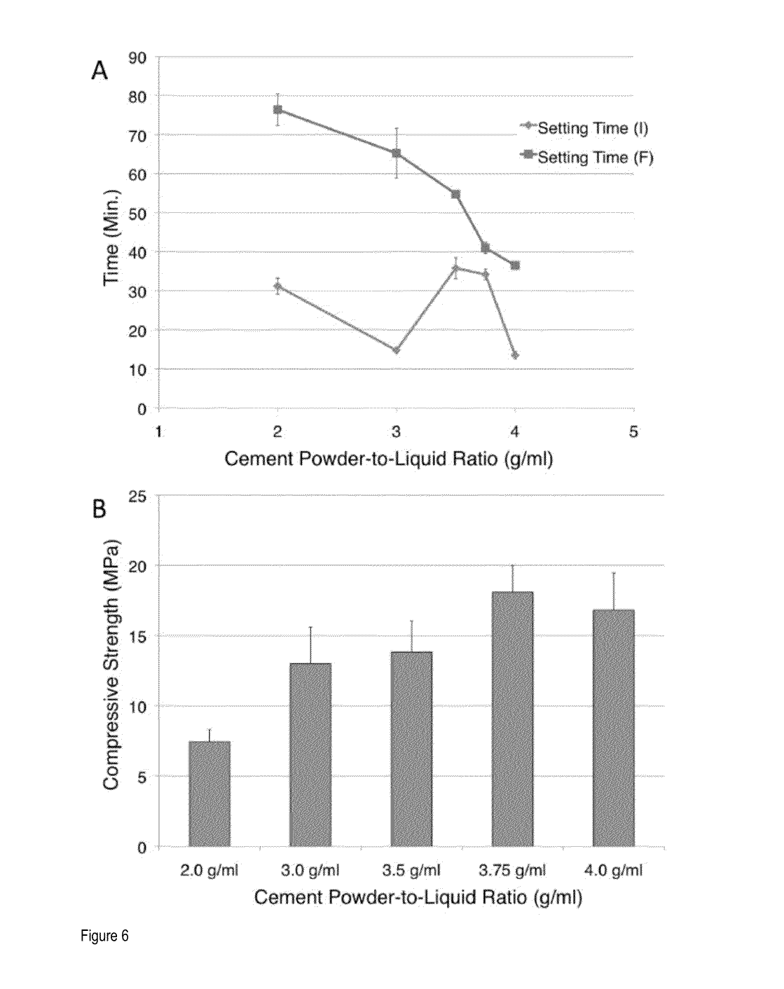

Powder-to-liquid ratio had far clearer results than the previous mixtures. Increases in P:L had a negative correlation with the setting times of the mixtures, most notably the final setting time, decreasing the time to setting by 50% between 2.0 and 4.0 g/ml (FIG. 6A). An effect on strength was also seen, with increases in compressive strength as P:L increased. The increase in strength was reasonably linear, with the strength of the 4.0 g/ml nearly 3 times that of the 2.0 g/ml material (FIG. 6B). Ease of handling changed dramatically with increases in P:L. Mixtures at 2.0 and 3.0 g/ml were liquid, which allowed them to be poured into the mould. This, however appeared to lead to poor distribution of the granules throughout these materials, leaving porous portions near the bottom where the low viscosity paste flowed out under the mould pieces. As P:L and the viscosity of the pastes increased, loading into the mould became easier and more homogenous distributions of the granules were produced with little to no external porosity seen. A P:L of 3.75 was found to have the best handling characteristics as it was easily applied to a sample implant by hand.

### Setting Time Optimization

Though the previous formulations showed excellent properties in strength and ease of handling, their setting times could, in some circumstances, be deemed less appropriate for the clinical setting. In an effort to decrease setting times, cured Crystacal Alpha K was ground up into powder seeds, which provided nucleation sites for the growth of the set material.

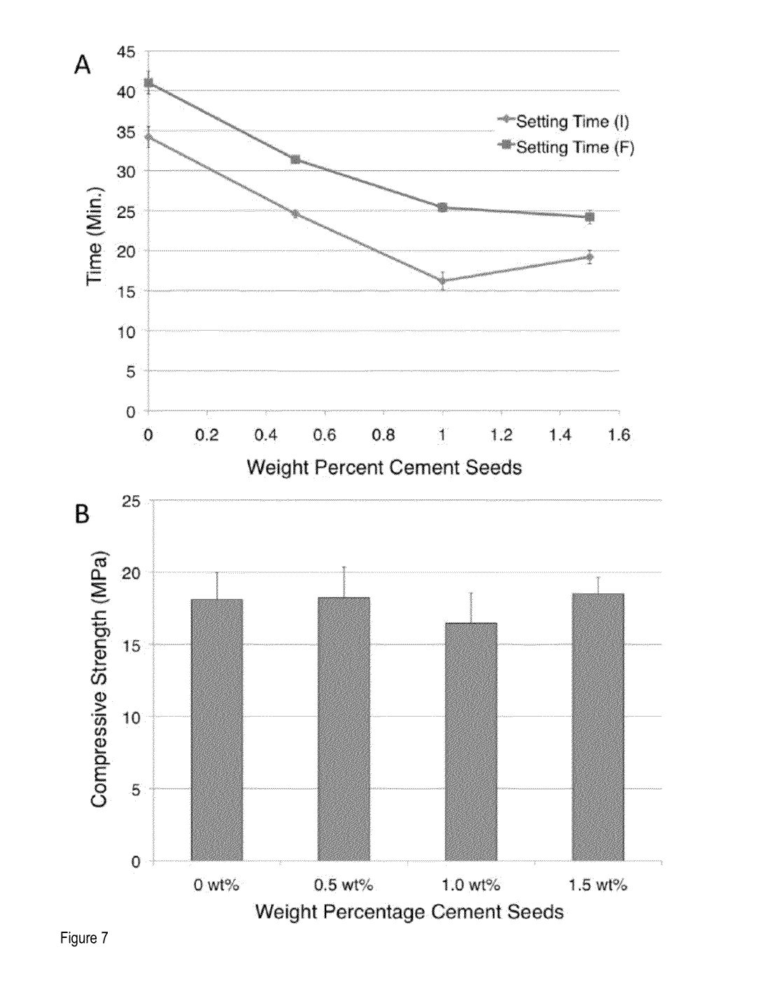

Increasing weight percentage of the seeds reduced the setting times of the material by over 15 minutes, keeping an even amount of time between initial and final setting (FIG. 7A). The compressive strength was not affected by the addition of cement seeds, remaining relatively even regardless of the weight percentage added (FIG. 7B). The seeds showed no effect on the mixing of the material, with pastes having the same viscosity and easy loading. While setting occurred more rapidly, the times are still within a range that will give the surgeon plenty of time to apply the material in situ.

### Optimal Composition

The optimal composition is presented in Table 2.

TABLE 2

| | |
|---|---|
| Granule Size (μm) | 212-500 |
| Granules (wt %) | 50 |
| Powder-to-Liquid Ratio (g/ml) | 3.75 |
| Cement Seeds (wt %) | 1.0 |
| Setting Time (Initial/Final) (min.) | 16/25 |
| Compressive Strength (MPa) | 16.5 |

### Example 2

### Bone Defect Healing Around Porous Titanium Implants

### Introduction

The healing potential of two different Ca-based materials in a canine implant model using porous titanium implants is reported below.

### Materials and Methods

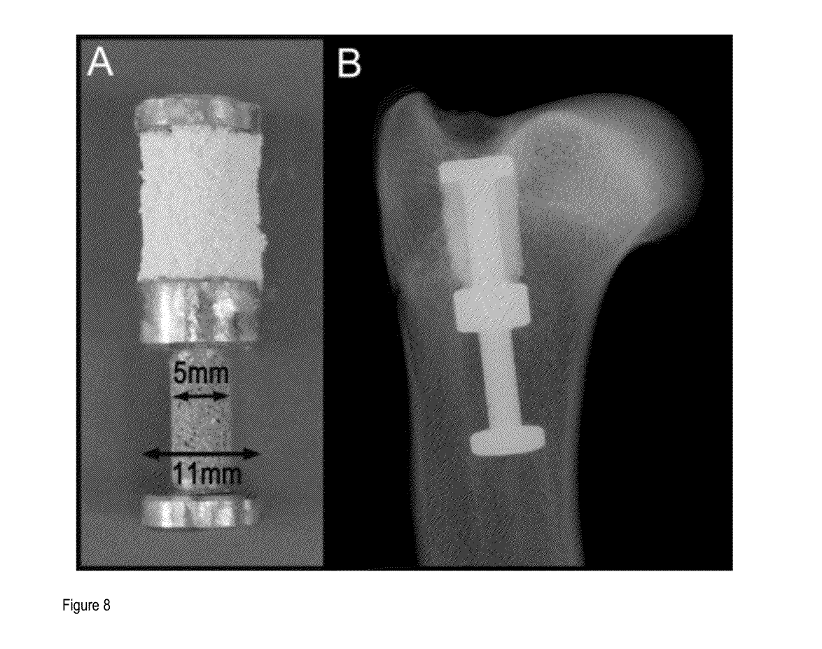

Gap-type intramedullary implants were fabricated from commercially pure titanium with a 5 mm diameter central porous rod and 11 mm diameter solid end and central spacers to create two separate 3 mm gap regions between host bone and porous metal implant (FIG. 8). The titanium foam core was 55% porous with an average pore size of 400 μm. One gap filling material was a commercial self-setting calcium phosphate material of nanocrystalline apatitic (hereinafter CaP) from Etex Corp, Mass. The composite of the invention (hereinafter CaS) was as described in Table 2 above. Adding sterile saline to the materials at the time of surgery produced compounds with handling characteristics conducive to molding and shaping into the implant gap regions. Prior to setting, the CaS compound was relatively soft and paste-like while the CaP compound was harder and more putty-like. The proximal 3 mm implant gaps were manually filled with either CaP or CaS, leaving the distal gaps empty as controls. Six dogs underwent bilateral surgery, each dog receiving one implant containing each material into the left or right proximal humerus (institutionally approved protocol). After 12 weeks, harvested to yield 6 sets of paired data from each animal comparing CaP with CaS. The humeri were scanned with a high voltage, high resolution microCT (μCT) scanner to obtain 18 μm thick serial images of the complete bone-implant construct. The resulting 1000 serial CT images of each gap were used to quantify the extent of resorption of the Ca-based materials and bone formation within the implant gaps, expressed as a volume percentage of the gap. Specimens were subsequently embedded in acrylic and undecalcified transverse serial sections were imaged with backscattered scanning electron microscopy (BSEM) to enable analysis of bone growth within the 3 mm gaps as well as within the porous implant regions. Statistical comparisons were made using paired and unpaired Student's t-tests and multiple two-level hierarchical models, with p≦0.05.

### Results

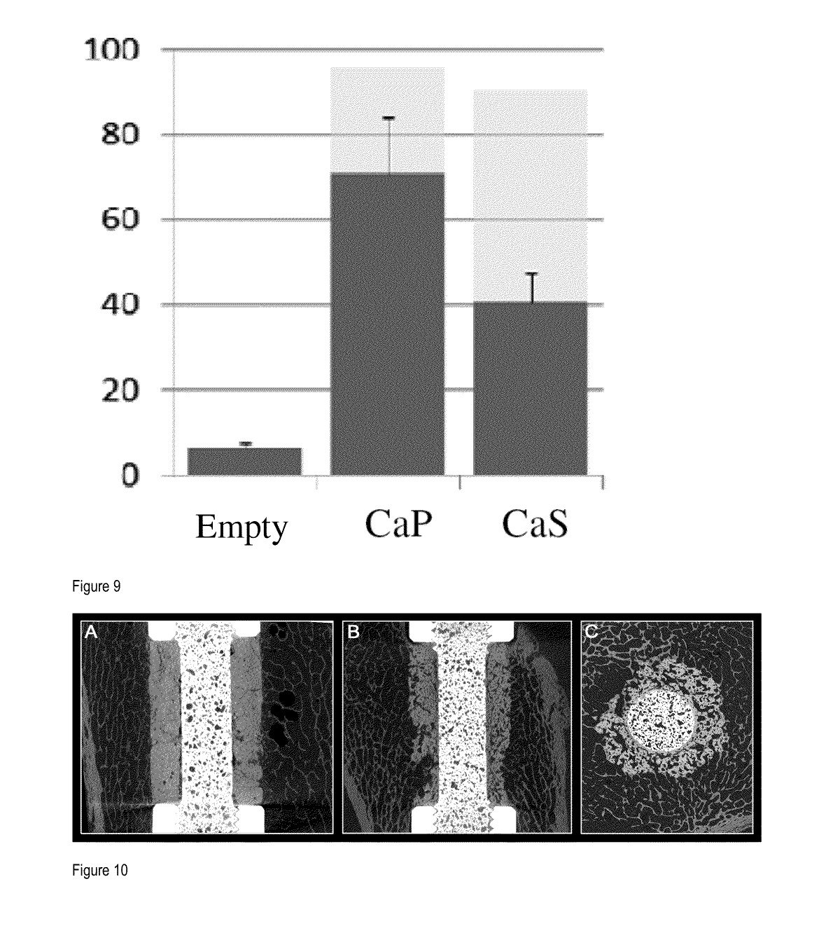

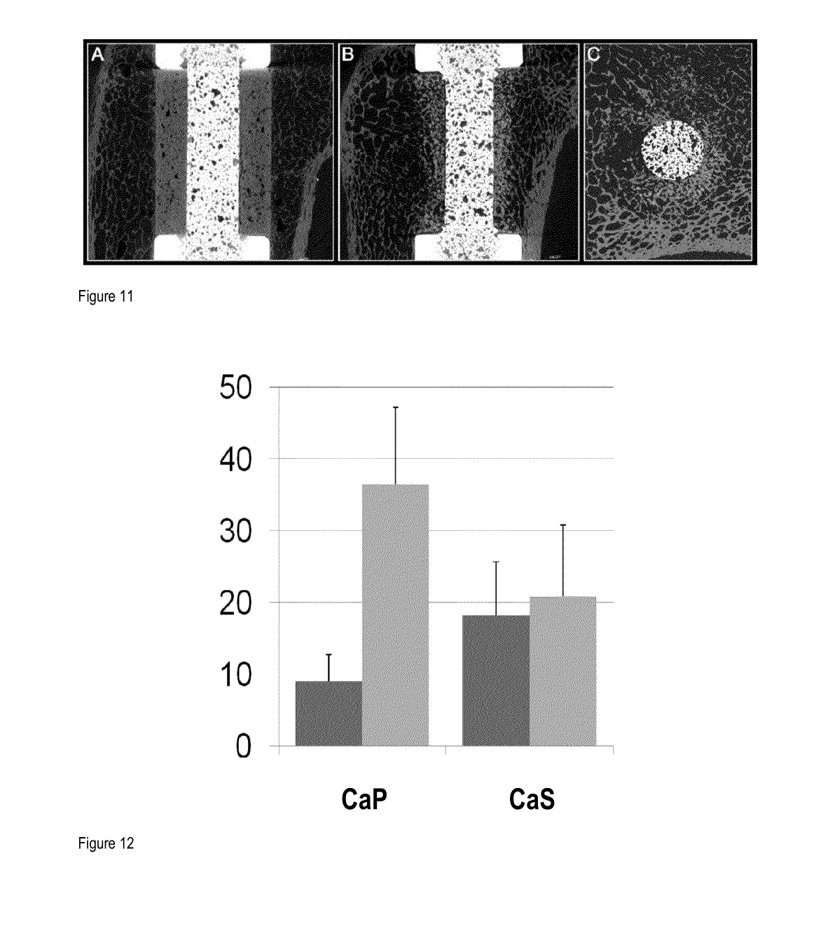

MicroCT quantified both native bone and residual CaS or CaP within the gaps, without discriminating one material from the other. Compared with time zero, the total material within CaP-filled gaps diminished by a mean volume of 25%±13% (FIG. 9). Compared with time zero, the total material within CaS-filled gaps diminished by a greater mean volume of 49%±7% (p=0.001). Compared with CaP at 12 weeks, the CaS material resorbed into a more porous scaffold within and on which new, interconnected trabeculae had formed in continuity with surrounding host bone (FIGS. 10, 11). Empty gaps were only 5%±1% filled at 12 weeks (p=0.001).

When these data were quantified (FIG. 12), it was apparent that bone in-growth was nearly double for the new composite. Similar results were obtained with demineralized human bone allografts.

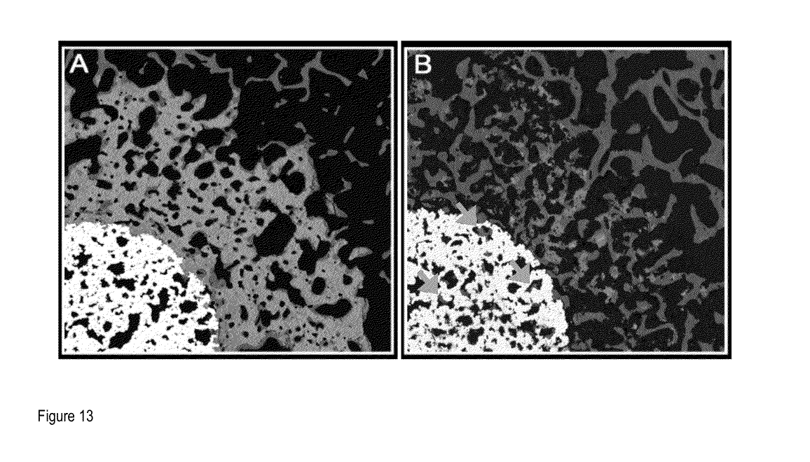

BSEM images more clearly showed that CaP-filled gaps remained predominantly filled by the material, with some new bone formation in and around the material pores (FIG. 13). In contrast, BSEM revealed that most of the CaS had resorbed by 12 weeks, with most of the gaps filled with dense bony trabeculae connecting the porous implant core with surrounding host bone (FIG. 13). CaP-filled implants showed more bone apposition at the porous implant perimeter than the CaS group (p=0.06) although the latter was associated with a greater mean extent of bone ingrowth (p=0.04). More specifically, the inventors observed 9±3 and 19±6% bone ingrowth for CaP and CaS, respectively.

### Discussion

Previous studies have shown that a 3 mm gap around a porous implant in the proximal humerus does not spontaneously heal with bone after 12 weeks. At time zero, both Ca-based materials filled almost the entire gap. By 12 weeks, both materials resorbed and new bond was evident within the gap, on and within the porous implant and in continuity with surrounding host bone. The CaS resorbed to a much greater extent, facilitating more gap filling with new bone.

The scope of the claims should not be limited by the preferred embodiments set forth in the examples, but should be given the broadest interpretation consistent with the description as a whole.

### REFERENCES

The present description refers to a number of documents, the content of which is herein incorporated by reference in their entirety. These documents include, but are not limited to, the following:
Beswick and Blom, Injury, 2011, S40-S46; and
Lim et al, Clin. Orthop. Relat. Res. 470, 2, 357-365.

1. A composite comprising monetite and calcium sulphate.

2. The composite of claim 1, wherein the monetite is in the form of granules in a matrix of calcium sulphate.

3. The composite of claim 2, where the granules range from about 10 to about 1500 μm in size.

4. The composite of claim 2, comprising between about 10 and about 80 wt % of granules, based on the total dry weight of the composite.

5. The composite of claim 1, further comprising an accelerant.

6. The composite of claim 5, wherein the accelerant comprises seeds of calcium sulphate.

7. The composite claim 1, wherein the calcium sulphate is calcium sulphate hemihydrate in powder form, and forms a powder matrix around the monetite.

8. The composite claim 1, wherein the calcium sulphate is calcium sulphate dihydrate or dehydrate in the form of a porous solid and forms a solid matrix around the monetite.

9. The composite claim 1, wherein the calcium sulphate is in powder form and wherein the calcium sulphate and the monetite are mixed with a curing liquid to form of a paste.

10. The composite of claim 9, wherein paste comprises between about 1.5 and about 4.0 g of calcium sulfate/monetite per ml of curing liquid.

11. The composite of claim 10, where the paste comprises between about 3.0 and about 4.0 g of calcium sulfate/monetite per ml of curing liquid

12. The composite of claim 10, wherein the curing liquid is saline or another non-toxic aqueous liquid.

13. A bone graft substitute made of the composite of claim 1.

14. A method for stimulating bone growth in a bone defect, the method comprising:
i. providing a composite according to claim 1,
ii. when needed, mixing the composite with a sterile curing liquid to form a paste,
iii. applying the composite in or near the defect, and
iv. when needed, allowing the composite to set.

15. A method for stimulating bone growth in a gap between a bone and an implant, in a gap between two bones, or in a gap in a single bone, the method comprising:
i. providing a composite according to claim 1,
ii. when needed, mixing the composite with a sterile curing liquid to form a paste,
iii. applying the composite in or near the gap,
iv. when needed, allowing the composite to set.

16. A method for enhancing healing of a bone-implant gap, the method comprising:
i. providing a composite according to claim 1,
ii. when needed, mixing the composite with a sterile curing liquid to form a paste,
iii. applying the composite in the gap,
iv. when needed, allowing the composite to set.

17. A method for enhancing osteointegration in a bone of a porous titanium bone implant, the method comprising:
i. providing a composite according to claim 1,
ii. when needed, mixing the composite with sterile curing liquid to form a paste,
iii. applying the composite in a gap between the bone and the implant,
iv. when needed, allowing the composite to set.

18. A method for promoting bone growth around an implant, the method comprising:
i. providing a composite according to claim 1,
ii. when needed, mixing the composite with sterile curing liquid to form a paste,
iii. applying the composite around the implant,
iv. when needed, allowing the mixture to set.

19. The method of claim 18, wherein the implant is a porous implant.
